(19) United States
(12) Patent Application Publication
(10) Pub. No.: US 2025/0312182 A1
(43) Pub. Date: Oct. 9, 2025

(54) SUPPORT CONFIGURED TO BE WORN BY A USER

(71) Applicants: Mark DRISCOLL, Ile Perrot (CA); Emeric BERNIER, Quebec (CA); Emilie DAVIGNON, Baie-D'Urfe (CA); Maria CALDERBANK, London (GB); Roseline THEROUX, Montreal (CA); Louis Hao Kiat TAN, Johor (MY)

(72) Inventors: Mark DRISCOLL, Ile Perrot (CA); Emeric BERNIER, Quebec (CA); Emilie DAVIGNON, Baie-D'Urfe (CA); Maria CALDERBANK, London (GB); Roseline THEROUX, Montreal (CA); Louis Hao Kiat TAN, Johor (MY)

(21) Appl. No.: 19/172,236

(22) Filed: Apr. 7, 2025

Related U.S. Application Data
(60) Provisional application No. 63/575,341, filed on Apr. 5, 2024.

(51) Int. Cl. A61F 5/02; A61F 5/03

(52) U.S. Cl. CPC A61F 5/028; A61F 5/03

(57) ABSTRACT

A support configured to be worn by a user. The support having a first anchor member configured to be removably secured to a first body part of the user and a strap configured to at least partially surround a second body part of the user. The support further includes a compression mechanism operably connected to the first anchor member and the strap. The compression mechanism is configured to modulate a parameter of the strap responsive to a first mechanical force being applied by the first anchor member to the compression mechanism in a first direction.

### CROSS-REFERENCE

This application claims priority to provisional application Ser. No. 63/575,341, filed on 5 Apr. 2024.

### TECHNICAL FIELD

The present technology relates to a support configured to be worn by a user, specifically but not exclusively a back support for providing support and stability to a spine of the user.

### BACKGROUND

Musculoskeletal disorders affect a person's mobility and dexterity leading to lower levels of well-being including physical discomfort and pain, emotional distress, reduced social engagement, decreased productivity or absences from work, medical costs, and financial strain. As the ageing population grows, the number of people impacted by musculoskeletal disorders are likely to increase.

Low back pain is a common musculoskeletal disorder affecting a large proportion of the population and is a leading cause of disability. A common cause for low back pain is spine instability. Spine instability often results in tissue strain and damage.

Back orthoses have been developed to prevent and treat spine instability. Back orthoses are configured to wrap around the abdomen, waist or torso and support and stabilize the spine by reducing movement and alleviating pressure of the affected area, relieving pain and discomfort, promoting healing, and enhancing general function of the spine. However, prolonged use of back orthoses may lead to muscle atrophy and discomfort due to its continuous engaged mechanism of action and restriction of the range of motion of the affected area.

Abdominal belts or exoskeletons, have been developed to assist mobility of the user during laborious activities such as heavy and/or frequent lifting, lowering the risk of muscle and/or joint strain and damage. These devices often compensate for the additional burden and strain on the area by redistributing the load or providing mechanical support to lift the load. However, these devices tend to be heavy and cumbersome, resulting in low compliance and limited effectiveness.

There is thus a desire to develop a support which overcomes the inconveniences present in the prior art.

### SUMMARY

It is an object of the present technology to ameliorate at least some of the inconveniences present in the prior art.

Broadly, there is provided a support configured to be worn by a user. In contrast to the prior art, the support is configured to selectively apply pressure to a body part of the user when triggered by the user through movement of another body part. The triggering body part is not the hands of the user in certain embodiments. Therefore, embodiments of the support of the present technology is selectively actuatable to apply selective pressure to the body part of the user, on an as needed basis.

In some embodiments, the support comprises a strap which is configured to tighten around an abdomen of the user, increasing intrabdominal pressure, responsive to the user bending forwards. As back injuries can be caused when lifting loads, the support is configured to be selectively supportive when the user bends forwards before carrying the load. In this respect, the support includes a portion that is worn by the user and that can modulate the strap when the user bends their back. The support is configured such that manual manipulation is not required for the selective modulation of the strap. Therefore, the user is able to keep their hands free whilst providing support to their spine. In other embodiments, the triggering body part movement may be a hand, arm, head or leg of the user. In some embodiments, the body part to which the pressure is selectively applied is an arm, leg, or chest of the user.

In some embodiments, the support provides users with a more comfortable experience while passively transferring forces and torques to the hips and lower body to assist motion, as well as stabilize and support the spine.

According to one aspect of the present technology, there is provided a support configured to be worn by a user. The support has a first anchor member configured to be removably secured to a first body part of the user and a strap configured to at least partially surround a second body part of the user. The support further includes a compression mechanism operably connected to the first anchor member and the strap. The compression mechanism is configured to modulate a parameter of the strap responsive to a first mechanical force applied by the first anchor member to the compression mechanism in a first direction. When the strap is worn around the second body part of the user, modulating the parameter of the strap may comprise causing a pressure to be applied to the second body part which can provide protective support to that body part.

In some embodiments, the first mechanical force is a tensile force, and the first direction is substantially perpendicular to a plane of the strap.

In some embodiments, the second body part is an abdomen of the user, and the strap is configured to surround at least a portion of the abdomen of the user.

In some embodiments, the strap is configured to be disposed around a thoracolumbar region of the abdomen.

In some embodiments, the first body part is an upper body of the user, and the first anchor member is configured to be removably secured to a portion of the upper body of the user, such that movement of the upper body of the user causes the first anchor member to move and apply the first mechanical force to the compression mechanism.

In some embodiments, the first anchor member has an elongate member configured to at least partially extend along a back of the user, the elongate member having a first end and a second end, the first end being operably connected to the compression mechanism; and a securing member connected to the second end of the elongate member and configured to be removably secured to an upper body of the user (e.g. shoulders, torso, etc.).

In some embodiments, the parameter of the strap comprises a distance or a separation between the first end and the second end of the strap. In some embodiments, the parameter of the strap is a circumference of the strap.

In some embodiments, the support further includes a second anchor member connected to the compression mechanism, such that the first anchor member is connected to the second anchor member via the compression mechanism, wherein the second anchor member is configured to be removably secured to a lower body portion of the user.

In some embodiments, the support further includes a first strap attachment connecting the first end of the strap to the compression mechanism at the first attachment point; a second strap attachment connecting the second end of the strap to the compression mechanism at the second attachment point; and a first anchor attachment connecting the first anchor member to the compression mechanism at a third attachment point, the third attachment point disposed in between the first and second attachment points.

In some embodiments, the compression mechanism includes a plurality of link members pivotably connected together to define a body such that as the link members move relative to one another, the body increases in a first dimension and simultaneously decreases in a second dimension, the first dimension being substantially perpendicular to the second dimension.

In some embodiments, the first dimension is a length of the body, and the second dimension is a width of the body.

In some embodiments, the plurality of links define a rhombus shape.

In some embodiments, the compression mechanism includes a cord extending between the first end of the strap and the second end of the strap, and the cord being operably connected to the first anchor member to move the first end and the second end of the strap relative to one another.

In some embodiments, the first end of the strap includes a first peg; the second end of the strap includes a second peg; and the cord is slidably received by the first peg and the second peg.

In some embodiments, the compression mechanism includes at least one pressurized inflatable cell disposed along the strap; the parameter of the strap is a pressure distribution of the at least one pressurized inflatable cell; and the at least one pressurized inflatable cell configured to re-distribute pressure of the at least one pressurized inflatable cell along the strap. In such embodiments, when the strap is worn around the body part of the user, inflating the at least one inflatable cell will cause pressure to be applied to the body part.

According to another aspect of the present technology, there is provided a support configured to be worn by a user. The support includes a first anchor member configured to be removably secured to a first body part of the user; a second anchor member configured to be removably secured to a second body part of the user; a strap configured to at least partially surround a third body part of the user, the strap having a first end and a second end; and a compression mechanism which is connected to the first and second ends of the strap at first and second attachment points, and connected to the first anchor member at a third attachment point. The compression mechanism is configured such that when the first anchor member applies a first mechanical force to the compression mechanism at the third attachment point, the first and second attachment points are configured to move relative to each other to modulate a circumference of the strap.

In some embodiments, the first mechanical force is a tensile force, and in response to the tensile force, the first and second attachment points move closer to one another.

In some embodiments, the compression mechanism is connected to the second anchor member at a fourth attachment point, and as the first anchor member applies the first mechanical force to the compression mechanism, the third and fourth attachment points are configured to move apart relative to each other.

According to another aspect of the present technology, there is provided a support configured to be worn by a user. The support includes a first anchor member configured to be removably secured to a first body part of the user. The support further includes a strap having a first end and a second end, the strap configured to, at least partially, surround a second body part of the user. A compression mechanism is operably connected to the first anchor member and the strap. The compression mechanism is configured to modulate distance or separation between the first end and the second end of the strap responsive to a first mechanical force applied by the first anchor member to the compression mechanism in a first direction.

In certain embodiments of the present technology, the support provides passively activated spinal support and stability by generating and/or increasing compression applied to the waist/abdomen of the user by the strap via the compression mechanism. Specifically, as the user performs a back flexion activity, the strap is configured to exert a tensile force onto the compression mechanism. In turn, the compression mechanism modulates the strap to apply compression to the waist/abdomen, thereby providing support and stabilization to the spine. By activating the back support only during user movement, the risk of muscle atrophy and discomfort from continuous engagement is minimized. The back support is designed to be compact and comfortable, enhancing user compliance and ensuring a more favorable user experience which, ultimately, may lead to improved outcomes. In certain embodiments, the support may function as one or more of: a posture support, a hip support and a lower back support.

In certain embodiments, the support can be used to assist rehabilitation from lower back pain. For example, the support can be used by sufferers of chronic and recurrent lower back pain. The support can be worn for prolonged periods whilst performing daily tasks.

In certain embodiments, the support may be used for one or more of: improved spinal stability which has been shown to reduce lower back pain; to improve posture and core strength gradually which can support rehabilitation from lower back pain (for example but not limited to post-operative spinal support, physical and occupational therapy, chronic lower back pain management, core muscle rehabilitation); support movement for preventing injury which could cause lower back pain; and assist lower back pain care and management.

The support is suitable for long-term and repetitive use in certain embodiments, as it provides on-demand support unlike existing acute devices. The support can be used by all demographics in daily tasks. Specific use can include one or more of: seniors for example in gardening, cleaning, balance, fall prevention, bladder control; support for people with disabilities; healthcare professionals in clinical daily tasks; obese individuals; athletic training; postpartum recovery; general support in awkward positions, lifting and crouching; manual work; and military use.

In certain embodiments, the support can be worn alongside, incorporated into, or attached to, clothing, exoskeletons, personal protective equipment, robotics, other equipment and the like. The support is also suitable to be worn under, over, or within clothing. The support can be incorporated into other orthosis and back supports. The support can also be adapted to be used on different parts of the body other than the back.

In this respect, the support has many uses such as, for example, supporting people with back pain across all demographics such as the aging population, healthcare professionals, manual workers, military, recreational and professional sports people, people with sedentary lifestyles such as long-haul truck drivers, office workers; people undergoing rehabilitation; people lifting heavy loads; people with improper crouching or lifting positions. The benefits are countless and include improved quality of life, improved mobility, an option to support at home rehabilitation, reduced reliance on pain killers, and the like.

In the context of the present specification, unless expressly provided otherwise, the words “first”, “second”, “third”, etc. have been used as adjectives only for the purpose of allowing for distinction between the nouns that they modify from one another, and not for the purpose of describing any particular relationship between those nouns.

It must be noted that, as used in this specification and the appended claims, the singular form “a”, “an” and “the” include plural referents unless the context clearly dictates otherwise.

As used herein, the term “and/or” is to be taken as specific disclosure of each of the two specified features or components with or without the other. For example “A and/or B” is to be taken as specific disclosure of each of (i) A, (ii) B and (iii) A and B, just as if each is set out individually herein.

Embodiments of the present technology each have at least one of the above-mentioned object and/or aspects, but do not necessarily have all of them. It should be understood that some aspects of the present technology that have resulted from attempting to attain the above-mentioned object may not satisfy this object and/or may satisfy other objects not specifically recited herein.

Additional and/or alternative features, aspects, and advantages of embodiments of the present technology will become apparent from the following description, the accompanying drawings, and the appended claims.

### BRIEF DESCRIPTION OF THE DRAWINGS

For a better understanding of the present technology, as well as other aspects and further features thereof, reference is made to the following description which is to be used in conjunction with the accompanying drawings, where:

FIG. 1 depicts a support of the present technology, when the support is worn by a user and viewed on a back of the user, according to non-limiting embodiments of the present technology;

FIG. 2 depicts the support of FIG. 1 viewed on a front of the user;

FIG. 3 depicts the support of FIG. 1 viewed on a side of the user;

FIG. 4 depicts the support of FIG. 1 showing a compression mechanism of the support in a neutral position;

FIG. 5 depicts the support of FIG. 1 showing a compression mechanism in a modulated position;

FIG. 6 depicts an alternative embodiment of a support of the present technology, when the support is worn by a user and viewed on a back of the user, according to non-limiting embodiments of the present technology;

FIG. 7 depicts a further alternative embodiment of a support of the present technology, when the support is worn by a user and viewed on a back of the user, according to non-limiting embodiments of the present technology; and

FIG. 8 depicts a side view of a portion of the support of FIG. 1.

### DETAILED DESCRIPTION

The present disclosure is not limited in its application to the details of construction and the arrangement of components set forth in the following description or illustrated in the drawings. The disclosure is capable of other embodiments and of being practiced or of being carried out in various ways. Also, the phraseology and terminology used herein is for the purpose of description and should not be regarded as limiting. The use of “including”, “comprising”, “having”, “containing”, “involving” and variations thereof herein, is meant to encompass the items listed thereafter as well as, optionally, additional items. In the following description, the same numerical references refer to similar elements.

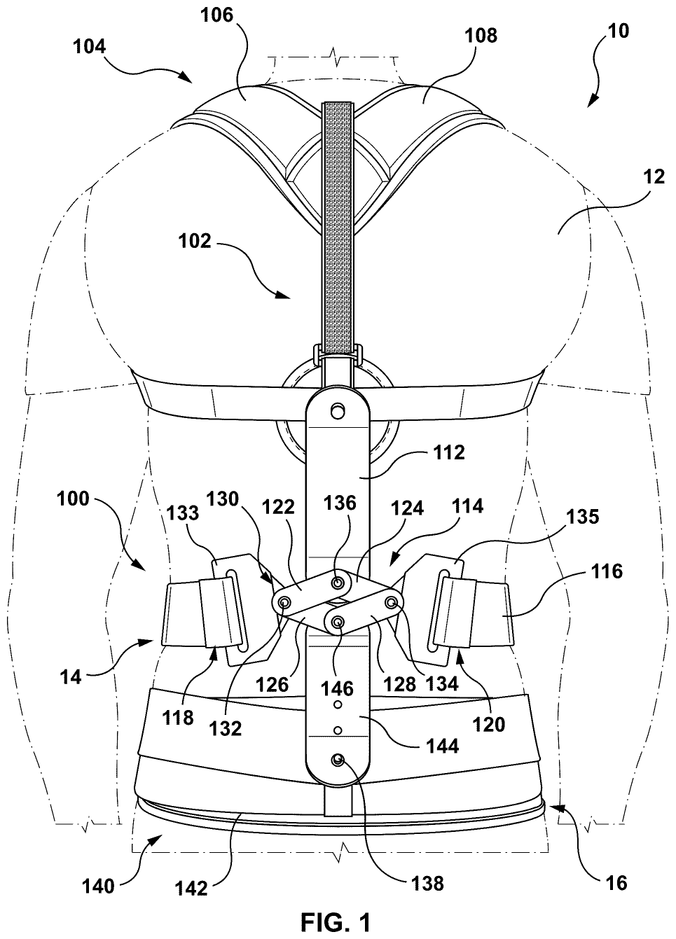

FIGS. 1 to 5 depict an embodiment of a support 100 configured to be worn by a user 10. In this embodiment, the support 100 is a back support. However, it is contemplated that the support 100 may be configured for other parts of the body in different embodiments.

In the present embodiment, the support 100 generates and/or increases compression experienced by the abdomen, low back, and/or sides of the user 10 to improve spinal stability and support during back flexion activities. The support 100 includes an anchor member 102 and a strap 116 operably connected to a compression mechanism 114 that modulates the strap 116 in response to a mechanical force applied by the anchor member 102. More specifically, as the user 10 performs a back flexion activity, the anchor member 102 applies a mechanical force to the compression mechanism 114. In response, the compression mechanism 114 modulates the strap 116 positioned around the user 10 such that the strap 116 applies a compressive force to a portion of the body of the user 10 surrounded by the strap 116 (e.g. abdomen, sides, lower back). With reference to FIG. 1, the anchor member 102 of the support 100 will now be described. In this embodiment, the anchor member 102 is an upper body anchor member 102 configured to be positioned on an upper body 12 of the user 10. The upper body anchor member 102 functions as a fixed support on the upper body 12 of the user 10. It is contemplated that the upper body anchor member 102 may be positioned at a different location on the user 10 in alternative embodiments.

The upper body anchor member 102 includes an upper body securing member 104 which is configured to be removably secured to shoulders of the user 10. The upper body securing member 104 includes two shoulder straps 106, 108, each configured to encircle a respective shoulder. The shoulder straps 106, 108 are composed of a flexible and/or semi-rigid material. In some embodiments, the shoulder straps 106, 108 are adjustable to provide comfort to the user 10, and to enable adjustability. This can permit the support to be a one-size-fits-all. In some embodiments, the straps 106, 108 may be composed of one or more of, synthetic rubber, silicone rubber, latex, and thermoplastic elastomer.

It is contemplated that, in alternative embodiments, the upper body securing member 104 may be configured to be removably secured to at least one of a base of a head and/or a neck of the user 10. In this instance, the upper body securing member 104 may provide additional benefits such as limiting cervical range of motion or assisting with cervical stability and support during cervical movements (e.g., cervical flexion). It is further contemplated that the upper body securing member 104 may be secured to a combination of upper body parts of the user 10. In alternative embodiments, the securing member 104 may be configured as a garment, such as a vest, to be worn by the user 10 which may distribute loads across a chest of the user 10. In some embodiments, the vest may be composed of one or more of cotton, spandex, synthetic fibers, bamboo, and nylon. In further alternative embodiments, the securing member 104 may be configured as a chest strap.

The upper body anchor member 102 is operably connected to the compression mechanism 114 via an elongate member 112. That is, a first end of the elongate member 112 is connected to the upper body anchor member 102 and a second end of the elongate member 112 is connected to the compression mechanism 114. The elongate member 112 extends partially along the back of the user 10. The elongate member 112 is configured to be positioned between the mid-to-lower back of the user 10, aligning with the spine of the user 10. In some embodiments, the elongate member 112 may be composed of a flexible, a semi-rigid, and/or a rigid material, for example a synthetic polymer such as polyethylene terephthalate or polytetrafluoroethylene. In other embodiments (not shown), the elongate member 112 and the anchor member 102 may be one-piece. Instead of a single anchor member 102, two or more anchor members can be provided.

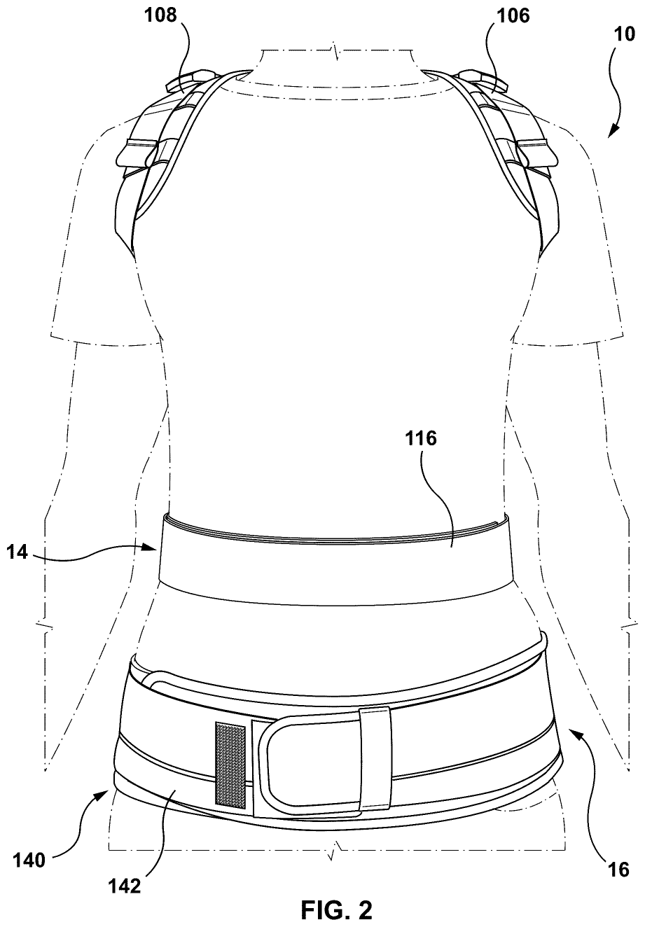

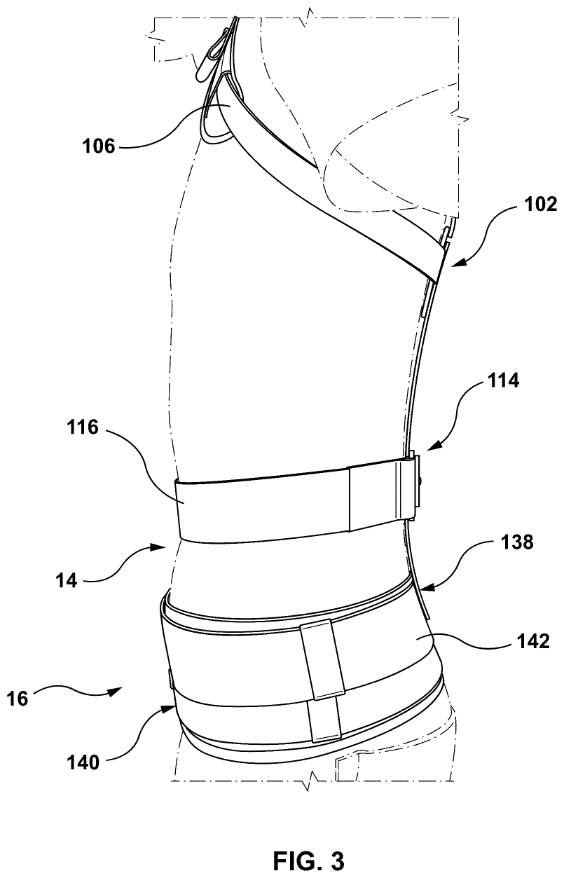

As discussed above, the support 100 further includes the strap 116 configured to surround a portion of the user 10. As seen in FIGS. 2 and 3, the strap 116 is configured to surround the abdomen/waist 14 of the user 10, for example, around a thoracolumbar region of the abdomen. At least one end 118, 120 (a first end 118 and a second end 120) of the strap 116 is connected to the compression mechanism 114, which will be described in further detail below. The strap 116 is composed of a flexible, semi-rigid and/or rigid material, such as a fabric e.g. webbing. In some embodiments, the strap 116 may be composed, at least in part, of one or more of, nylon webbing, synthetic rubber, silicone rubber, latex and thermoplastic elastomer. The strap 116 may include a foam padding. The strap 116 may include a component to modulate a rigidity of the strap 116 in one or more regions. In some embodiments, a plurality of straps 116 having different tensile properties and hence different compressive effects may be provided and a given strap 116, or a combination of straps 116, may be selected based on a desired compression to the user 10. In this respect, the strap 116 is removably attachable to the support 100.

With specific reference to FIGS. 1, 4 and 5, the compression mechanism 114 will now be described in detail. Broadly, the compression mechanism 114 is configured to modulate a separation between the ends 118, 120 of the strap 116 when force is applied to the compression mechanism. In certain embodiments, the compression mechanism 114 is configured to modulate the distance between the ends 118, 120 of the strap when the force is applied in a direction substantially transversely to a direction of the movement between the ends 118, 120.

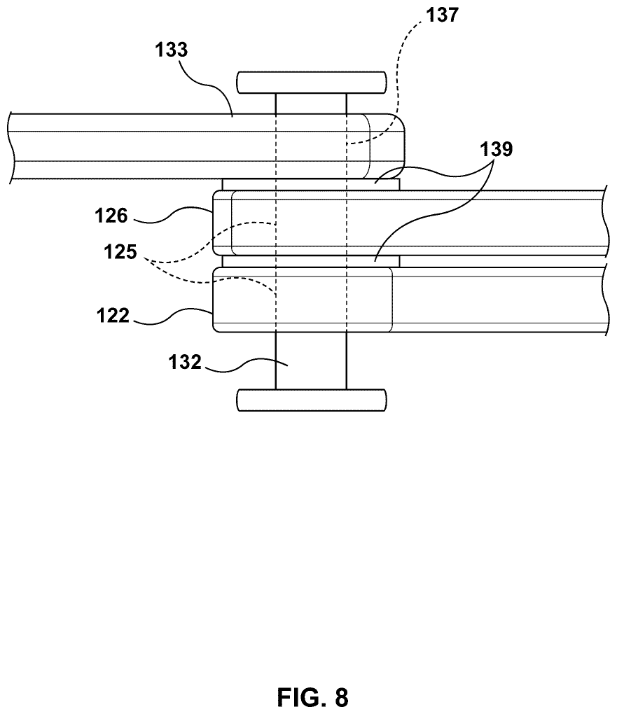

In this embodiment, the compression mechanism 114 includes a plurality of links 122, 124, 126, 128. The links 122, 124, 126, 128 are pivotably connected to one another to define a body 130. Specifically, ends of each link are connected to another link. In this embodiment, the body 130 is rhomboid-shaped. However, it is contemplated that the body 130 may define another shape in alternative embodiments. The links 122, 124, 126, 128 are pivotably connected, via fasteners such as pins, such that the links 122, 124, 126, 128 can move relative to one another. As depicted in FIG. 8, each link 122, 124, 126, 128 defines an aperture 125 that receives a corresponding pin. In particular, the links 122, 124, 126, 128 move relative to one another to increase a parameter of the body 130 (such as length) in a first dimension, while simultaneously decreasing a parameter of the body 130 (such as width) in a second dimension. It is contemplated that other fasteners to connect the links 122, 124, 126, 128 may be used and enable relative movement between the links 122, 124, 126, 128. In some embodiments, the links 122, 124, 126, 128 are composed of a rigid material and/or semi-rigid material, such as a metal and/or metal alloy (for example, steel or aluminum), and/or rigid plastic (for example, polyvinyl chloride, high density polyethylene, acrylonitrile butadiene, polycarbonate, and polypropylene).

The compression mechanism 114 has a plurality of attachment points 132, 134, 136 for connecting the ends 118, 120 of the strap 116 and the second end of the elongate member 112, respectively. In other words, the compression mechanism 114 includes two strap attachment points 132, 134. One strap attachment point 132 is connected to the first end 118 of the strap 116 via a first buckle 133. The other strap attachment point 134 is connected to the second end 120 of the strap 116 via a second buckle 135. The first and second ends 118, 120 of the strap 116 may be detachably connected to the first and second buckles 133, 135, respectively. The two strap attachment points 132, 134 are oppositely facing one another. An upper anchor attachment point 136, disposed in between the two strap attachment points 132, 134, operably connects to the upper body anchor member 102 via the elongate member 112. In this embodiment, each of the attachment points 132, 134, 136 correspond to the pins 132, 134, 136 which pivotably connect the links 122, 124, 126, 128 to one another. With reference to FIG. 8, the first buckle 133 defines a buckle aperture 137. The pin 132 extends through the buckle aperture 137 and the aperture 125 defined in the corresponding links 122, 126. In this embodiment, washers 139 are disposed in between the first buckle 133 and link 126, as well as in between link 126 and link 122. It is noted that the second buckle 135 has a similar configuration to the first buckle 133. When the strap 116 is connected to the compression mechanism 114, two strap attachment points 132, 134 are aligned with a longitudinal axis of the strap 116. In other embodiments (not shown), the strap attachment points 132, 134 may have any other alignment, for example to accommodate for, and be positioned below, a pregnant woman's belly.

It is contemplated that, in alternative embodiments, instead of a pin, the buckles 133, 135 may include a rod configured to extend through the apertures 125 of the links 122, 124, 126, 128. It is to be understood that, in other embodiments, the attachment points 132, 134, 136 and the pins pivotably connecting the links 122, 124, 126, 128 may be separate from one another. It is appreciated that the ends 118, 120 of the strap 116 and the second end of the elongate member 112 may be connected to the compression mechanism 114 by other fastening means. For example, in other embodiments (not shown), the first and second buckles 133, 135 may be omitted. In such embodiments, the strap attachment points 132, 134 are connected directly to the first and second ends 118, 120 of the strap 116 using, for example, pins.

With continued reference to FIGS. 1 to 3, the support 100 further includes a lower body anchor member 138 configured to be positioned at a lower body 16 of the user 10, such as aligned with a waist or hips of the user 10. In other words, the compression mechanism 114 is disposed in between the upper and the lower anchor members 102, 138. The lower body anchor member 138 is operably connected to the compression mechanism 114. The lower body anchor member 138 functions as a fixed support on the lower body 16 of the user 10. In use, the lower body anchor member 138 can prevent the compression mechanism from moving upwards when the user bends forward.

In this embodiment, the lower body anchor member 138 includes a lower body securing member 140 which is configured to be removably secured to the lower body 16 of the user 10. The lower body securing member 140 includes a waist strap 142 configured to encircle the waist or hips of the user 10, for example at sacrum level. In some embodiments, the lower body securing member 140 may further include weights to hold the waist strap 142 in place, thereby mitigating slipping of the waist strap 142 during back flexion.

It is contemplated that, in alternative embodiments, the lower body securing member 140 may, alternatively or additionally, be configured to be removably secured to at least one of the thigh, ankle, calf, and foot of the user 10. In this instance, the lower body securing member 140 may further facilitate distributing longitudinal pressure over the respective lower body part. For example, the elongate member 144 may extend to the thigh whilst omitting the lower body securing member 140. In alternative embodiments, the lower body securing member 140 may be configured as a garment to be worn by the user 10. In further alternative embodiments, the lower body securing member 140 may have an interface to mechanically couple the upper and the lower body securing members 104, 140. In some embodiments, the garment may be composed of one or more of cotton, spandex, synthetic fibers, bamboo, and nylon.

With specific reference to FIGS. 1, 4, and 5, the lower body anchor member 138 is operably connected to the compression mechanism 114 via an elongate member 144. That is, a first end of the elongate member 144 is connected to the lower body anchor member 138 and a second end of the lower body anchor member 144 is connected to the compression mechanism 114. The elongate member 144 extends partially along the back of the user 10. The elongate member 144 is positioned on the lower back of the user 10, aligning with the spine of the user 10. In some embodiments, the elongate member 144 may be composed of a flexible, a semi-rigid, and/or a rigid material. It is contemplated that, in alternative embodiments, the lower body anchor member 138, the lower body securing member 140, the waist strap 142, and the elongate member 144 may be omitted.

In this embodiment, the compression mechanism 114 further includes a lower attachment point 146 for connecting the second end of the elongate member 144. The lower attachment point 146 is disposed in between the two strap attachment points 132, 134 and opposite to that of the upper attachment point 136. The lower attachment point 146 operably connects the compression mechanism 114 to the lower body anchor member 138, via the elongate member 144. In this embodiment, the lower attachment point 146 includes a fastener, such as a pin, for connecting the elongate member 144 to the waist strap 142. It is to be understood that the lower attachment point 146 and the pin connecting the links 126, 128 may be separate from one another in other embodiments. It is appreciated that the second end of the elongate member 144 may be connected to the compression mechanism 114 by other fastening means.

It will be appreciated that the strap is made of any suitable material that is comfortable to the user yet has a tensile property (e.g., elastic modulus, stiffness, etc.) that can provide a desired pressure to the user. Similarly, portions of the support 100 that are configured to cooperate together to apply a tensile force to the compression mechanism have a tensile property that is comfortable to the user yet not too elastic such that a bending motion can provide enough of a tensile force to the compression mechanism to actuate the compression mechanism. In certain embodiments, the support 100 is configured to apply an intrabdominal pressure to the user, when actuated, between a range of 0 and about 300 mmHg, or about 5 to about 300 mmHg.

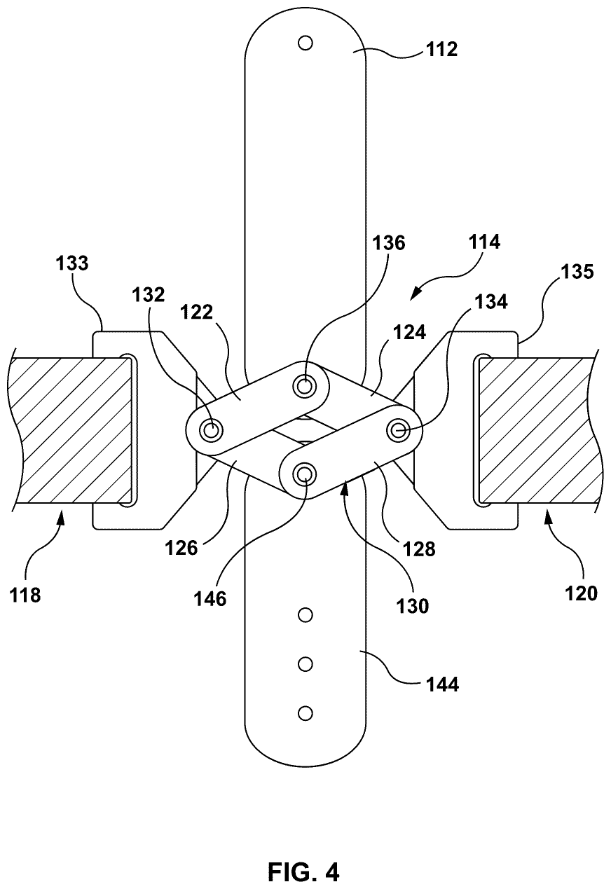

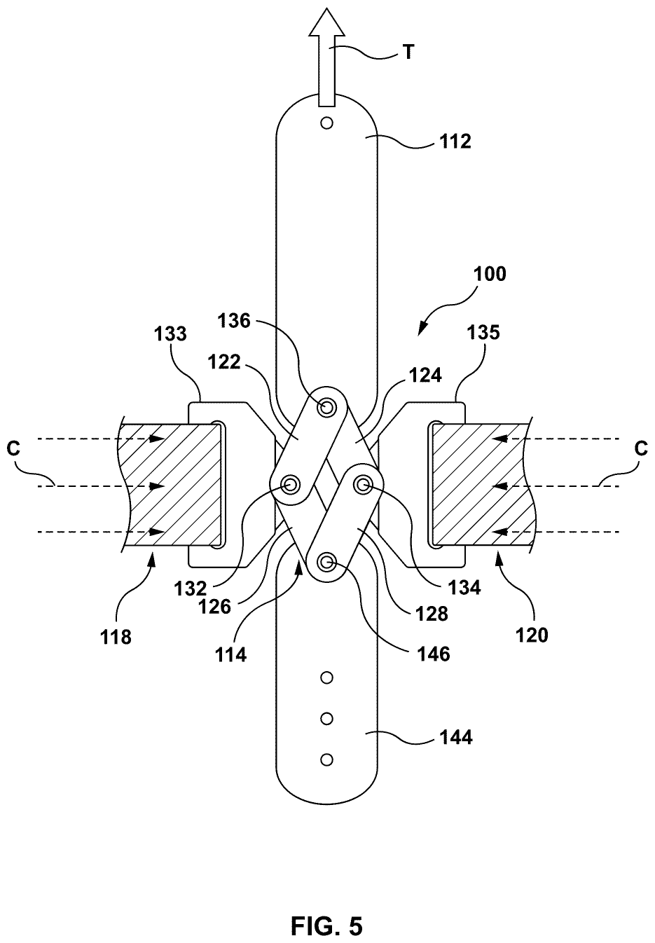

With specific reference to FIGS. 4 and 5, the use and functioning of the support 100 will now be described in detail. As discussed above, the body 130 of the compression mechanism 114 is configured to move bidirectionally, that is a parameter of the body 130 increases in one dimension (e.g. length) whilst a transverse parameter (e.g. width) reduces in response to the mechanical force being applied by the upper body anchor member 104. In some embodiments, the dimension which is increased may comprise the width (e.g. radial expansion of the strap 116) and the dimension which is reduced in response is the length (e.g. distance of the shoulder straps 106, 108 from the compression mechanism 114).

As the user 10 undergoes back flexion, the upper body anchor member 102 applies a tensile force (denoted by T in FIG. 5) to the compression mechanism 114. More specifically, the upper body anchor member 104 exerts the tensile force T to the elongate member 112 which transfers the tensile force to the upper attachment point 136 of the compression mechanism 114. The lower body anchor member 138, operably connected to the compression mechanism 114 via the elongate member 144, retains the position of the lower attachment point 146. In other words, the lower attachment point 146 is substantially held in place when the tensile force T is applied. Thus, the upper attachment point 136 moves away from the lower attachment point 146, elongating the body 130. The direction of elongation is along the back of the user when the user is wearing the support 100. Simultaneously, the two strap attachment points 132, 134 move closer together (denoted by C in FIG. 5), thereby reducing the width of the body 130. This causes the distance between the two strap attachment points 132, 134 to also be reduced. As the two strap attachment points 132, 134 move closer together, the ends 118, 120 of the strap 116 are moved closer together. This causes the strap 116 to tighten, applying a compressive force to the body parts (e.g., the abdomen, lower back, and/or sides) of the user 10 that are encircled by the strap 116. In other words, due to the rhomboid-shape of the body 130, the tensile force T applied to the body 130 is translated to a compressive force applied by the strap 116. Thus, the support 100 provides the user 10 with a passively activated mechanical solution to support and stabilize the spine of the user 10 during back flexion by generating and/or increasing compression applied by the strap 116 to the user 10.

Conversely, as the user 10 returns to a neutral position (that is, the position depicted in FIGS. 1 to 3), the compression mechanism 114 also returns to its neutral position, removing and/or reducing the compression applied by the strap 116.

It is contemplated that, in an alternative embodiment, the support 100 may be positioned to provide support and stability to the spine during other back movements, such as back extension. In this instance, the compression mechanism 114 may be positioned on the abdomen of the user 10 (i.e., rotated by 180 degrees to the positioning shown in FIGS. 1 to 3).

In further alternative embodiments, as mentioned above, the support 100 may be configured such that when the strap 116 is caused to expand radially, the upper and/or lower body anchors are caused to shorten thereby providing posture correction.

In other embodiments (not shown), any one or more components of the support may be adapted in any manner for tuning the support to a given use. For example, it may be desired to tune a degree of the radial compression following the user flexion. In this respect, the support may include one or more members for modulating the resultant compression, such as a spring. The resultant compression can also be modulated or tuned by modifying any of the straps of the support, for example the strap 116, such as by adapting an elasticity and/or changing a number of the straps, etc. Other modifications will be apparent to persons of skill in the art.

It will be appreciated, that in some embodiments the lower body securing member 140 can create the tensile force to drive the compression mechanism 114 (e.g. if the user kneels or squats). In further alternative embodiments, the support 100 may be positioned on any other body part of the user, such as a leg, arm, chest etc.

The support 100 may be configured to be adjustable so as to be suitable for use with many different users. In this respect, the support 100 may have one or more components that are adjustable, such as one or more of the strap 116, the shoulder straps 106, 108, the elongate member 112, the lower body securing member 140, waist strap 142, elongate member 144, etc.

Alternatively, the support 100 may be customized to a given user 10. In this respect, the support can be designed based on measurements of the user 10.

It is contemplated that, in alternative embodiments, the compression mechanism 114 may instead be material defining the body 130 (i.e. having a one-piece construction). In this instance, the material is operably connected to the upper anchor member 102, strap 116, and lower body anchor member 138. The material is configured such that modulation of the material in one direction causes simultaneous modulation in another direction. For example, increasing a parameter of the body 130 (such as length) in a first dimension, causes simultaneous decrease of a parameter of the body 130 (such as width) in a second dimension. For example, in some cases, the material may comprise a resilient material or a material with shape memory properties.

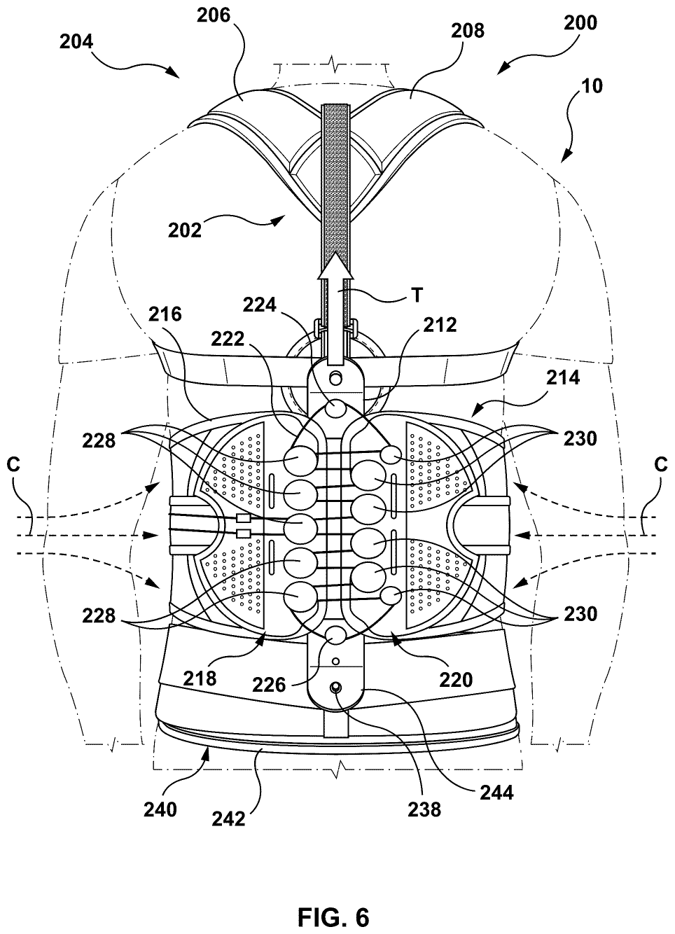

With reference to FIG. 6, an alternative embodiment of a support 200 will now be described. The support 200 includes an upper body anchor member 202 (including an upper body securing member 204 having shoulder straps 206, 208), an elongate member 212, a strap 216 (having a first end 218 and a second end 220), a lower body anchor member 238 (including a lower body securing member 240 having a waist strap 242), and an elongate member 244 which are similar to those of the support 100, and therefore will not be described in further detail.

In this embodiment, the support 200 includes a compression mechanism 214 having a cord 222 extending between the first and second ends 218, 220 of the strap 216. The cord 222 is operably connected to the upper and the lower body anchor members 202, 238 via the elongate members 212, 244 respectively. More specifically, each of the elongate members 212, 244 include an anchor peg 224, 226 for slidably receiving the cord 222.

The compression mechanism 214 further includes two sets of strap pegs 228, 230 (a first set of strap pegs 228 and a second set of strap pegs 230). The first set of strap pegs 228 is disposed along the first end 218 of the strap 216 and the second set of strap pegs 230 is disposed along the second end 220 of the strap 216. The cord 222 is slidably received by the first and the second set of strap pegs 228, 230. The cord 222 extends between the first and the second set of strap pegs 228, 230 forming a corset-type arrangement. Although, in FIG. 6, a plurality of strap pegs 228, 230 are depicted, it is to be understood that each set of strap pegs 228, 230 may include any number of pegs (e.g., one or more pegs) positioned on each end 218, 220 of the strap 216.

As described above, the cord 222 is slidably received in the anchor peg 224 associated with the upper body anchor member 202 such that, as the user 10 undergoes back flexion, a tensile force (denoted as T in FIG. 6) is applied to the anchor peg 224 which applies an upward force to the cord 222. The lower body anchor member 226 acts as a fixed lower support for the associated anchor peg 226 such that the anchor peg 226 remains at its position on the lower body of the user 10. Therefore, as the tensile force T is applied, the anchor pegs 224, 226 move apart from one another. In turn, the cord 222 is pulled upwards with the anchor member 224, causing the first and the second set of strap pegs 228, 230 to move closer to one another (denoted by C in FIG. 6). As a result, as the tensile force T is applied, the ends 218, 220 of the strap 216 are cinched closer to one another causing the strap 216 to tighten and apply a compressive force to a portion of the body of the user 10 surrounded by the strap 116 (e.g. abdomen, sides, lower back).

Conversely, as the user 10 returns to a neutral position (that is, the position depicted in FIG. 6), the compression mechanism 114 also returns to its neutral position, removing and/or reducing the compression applied by the strap 116.

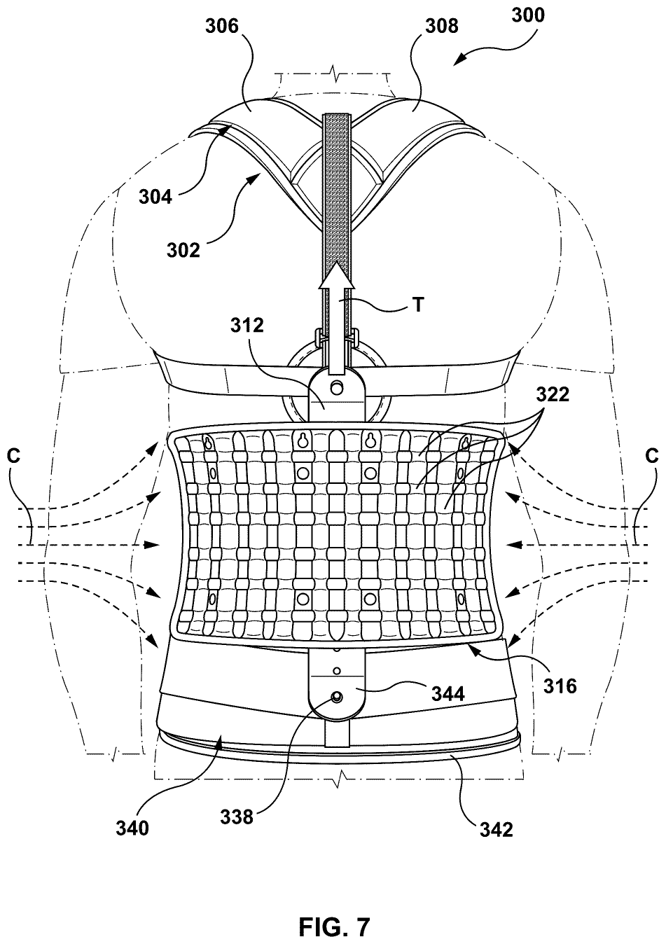

With reference to FIG. 7, an alternative embodiment of a support 300 will now be described. The support 300 includes an upper body anchor member 302 (including an upper body securing member 304 having shoulder straps 306, 308), an elongate member 312, a strap 316 having a first and second end (not shown), a lower body anchor member 338 (including a lower body securing member 340 having a waist strap 342), and an elongate member 344 which are similar to those of the support 100, and therefore will not be described in further detail.

In this embodiment, the compression mechanism 314 includes a plurality of pressurized inflatable cells 322 in fluid communication with one another. The pressurized cells 322 are distributed along the strap 316. As the user 10 undergoes back flexion, a tensile force (denoted by T in FIG. 7) is applied to the compression mechanism 314 via the upper body anchor member 302. Similar to the support 100, 200, the lower body anchor member 338 acts as a lower fixed support. As a result, some of the pressurized inflatable cells 322 are put under tension (i.e., those cells 322 which are close to the applied tensile force T) which redistributes the pressure to adjacent cells (i.e., those around the abdomen, lower back, and sides of the user 10), leading to a compressive force being applied by the strap 316 to the user 10. Although, in FIG. 7, a plurality of pressurized inflatable cells 322 are depicted, it is to be understood that any number (e.g., one or more) and distribution of pressurized cells 322 along the strap 316 may be used. Furthermore, there may be provided a pump or the like to pressurize the cells 322.

Conversely, as the user 10 returns to a neutral position (such as the position depicted in FIG. 7), the pressure within the pressurized cells 322 are redistributed to the neutral distribution.

It is contemplated, in an alternative embodiment, that the compression mechanism 314 may inflate and/or deflate the pressurized cells 322 in response to back flexion or other triggering activities. The disclosed embodiments of the support 100, 200, 300 include, but are not limited to, providing the user 10 with a passively activated mechanical advantage in response to the user 10 bending forward. In other words, the support 100, 200, 300 does not require hand activation, allowing the user 10 to keep their hands free while still providing spinal support during back flexion activities. Furthermore, in some embodiments, the support 100, 200, 300 may not be continuously engaged or activated. Rather, the support 100, 200, 300 is only activated during back flexion which mitigates risk of discomfort and/or muscle atrophy. In other embodiments, the support 100, 200, 300 may be positioned tightly on the user 10 to provide constant compression. In this instance, during back flexion, the user 10 would feel an increase in compression. Additionally, the support 100, 200, 300 has a comfortable and compact design which may lead to an increase in user 10 compliance, and an overall increase in effectiveness of the support 100, 200, 300.

### EXAMPLE

Fourteen participants with chronic non-specific low back pain and eighteen gender-matched healthy controls undertook controlled movement tests with and without the support 100 (described further in Bernier, E et al. Clinical Biomechanics, Volume 122, 106414, the contents of which are herein incorporated in their entirety).

It was found that the support increased intra-abdominal pressure in both groups during various functional tasks, more notably in active flexion and lifting tasks (up to +43%). The support 100 also contributed to decreasing lumbar range of motion during guided flexion (−18 to −37%, except at the lumbosacral junction in the low back pain group), emphasizing its potential impact in limiting excessive spinal movement. Muscle activity assessments revealed decreased activation during active flexion and lifting movements while wearing the support 100, suggesting the possibility to assist trunk stabilization without the corresponding antagonistic muscle activation and associated compressive load on the spine. These effects could help workers to maintain their activities in the workplace and help workers suffering from low back pain to gradually reintegrate work or physical activities, contributing to better overall management of back health.

In view of the various disclosures related to the support 100, 200, 300, it will be understood that, although the embodiments presented herein have been described with reference to specific features and structures, it is clear that various modifications and combinations may be made without departing from such disclosures. The specifications and drawings are, accordingly, to be regarded simply as an illustration of the discussed implementations or embodiments and their principles as defined by the appended claims, and are contemplated to cover any and all modifications, variations, combinations, or equivalents that fall within the scope of the present disclosure.

What is claimed is:

1. A support configured to be worn by a user, the support comprising:
a first anchor member configured to be removably secured to a first body part of the user;
a strap configured to at least partially surround a second body part of the user; and
a compression mechanism operably connected to the first anchor member and the strap,
wherein the compression mechanism is configured to modulate a parameter of the strap responsive to a first mechanical force applied by the first anchor member to the compression mechanism in a first direction.

2. The support of claim 1, wherein the first mechanical force is a tensile force, and the first direction is substantially perpendicular to a plane of the strap.

3. The support of claim 1, wherein the second body part is an abdomen of the user, and the strap is configured to surround at least a portion of the abdomen of the user.

4. The support of claim 3, wherein the strap is configured to be disposed around a thoracolumbar region of the abdomen.

5. The support of claim 1, wherein the first body part is an upper body of the user, and the first anchor member is configured to be removably secured to a portion of the upper body of the user, such that movement of the upper body of the user causes the first anchor member to move and apply the first mechanical force to the compression mechanism.

6. The support of claim 1, wherein the first anchor member comprises:
an elongate member configured to at least partially extend along a back of the user, the elongate member having a first end and a second end, the first end being operably connected to the compression mechanism; and
a securing member connected to the second end of the elongate member and configured to be removably secured to an upper body of the user.

7. The support of claim 6, wherein the parameter of the strap comprises a distance between the first end and the second end of the strap.

8. The support of claim 1, further comprising a second anchor member connected to the compression mechanism, such that the first anchor member is connected to the second anchor member via the compression mechanism, wherein the second anchor member is configured to be removably secured to a lower body portion of the user.

9. The support of claim 8, wherein the first and the second anchor members are configured such that a relative change in position between the first and the second anchor members causes the compression mechanism to modulate the parameter of the strap.

10. The support of claim 1, wherein the strap has a first end and a second end attached to respective first and second attachment points of the compression mechanism, wherein the compression mechanism is configured to move the first and second ends of the strap closer together and/or further apart to modulate the parameter of the strap.

11. The support of claim 10, further comprising:
a first strap attachment connecting the first end of the strap to the compression mechanism at the first attachment point;
a second strap attachment connecting the second end of the strap to the compression mechanism at the second attachment point; and
a first anchor attachment connecting the first anchor member to the compression mechanism at a third attachment point, the third attachment point disposed in between the first and second attachment points.

12. The support of claim 1, wherein the compression mechanism comprises a plurality of link members pivotably connected together to define a body such that as the link members move relative to one another, the body increases in a first dimension and simultaneously decreases in a second dimension, the first dimension being substantially perpendicular to the second dimension.

13. The support of claim 12, wherein the first dimension is a length of the body, and the second dimension is a width of the body.

14. The support of claim 12, wherein the plurality of links define a rhombus shape.

15. The support of claim 1, wherein:
the compression mechanism comprises a cord extending between a first end of the strap and a second end of the strap, and the cord being operably connected to the first anchor member to move the first end and the second end of the strap relative to one another.

16. The support of claim 15, wherein:
the first end of the strap includes a first peg;
the second end of the strap includes a second peg; and
the cord is slidably received by the first peg and the second peg.

17. The support of claim 1, wherein:
the compression mechanism comprises at least one pressurized inflatable cell disposed along the strap;
the parameter of the strap is a pressure distribution of the at least one pressurized inflatable cell; and
in response to the first mechanical force being applied by the first anchor member, the at least one pressurized inflatable cell re-distributes the pressure distribution along the strap.

18. A support configured to be worn by a user, the support comprising:
a first anchor member configured to be removably secured to a first body part of the user;
a second anchor member configured to be removably secured to a second body part of the user;
a strap configured to at least partially surround a third body part of the user, the strap having a first end and a second end;
a compression mechanism which is connected to the first and second ends of the strap at first and second attachment points, and connected to the first anchor member at a third attachment point, and
wherein the compression mechanism is configured such that when the first anchor member applies a first mechanical force to the compression mechanism at the third attachment point, the first and second attachment points are configured to move relative to each other to modulate a circumference of the strap.

19. The support of claim 18, wherein the first mechanical force is a tensile force, and in response to the tensile force, the first and second attachment points move closer to one another.

20. The support of claim 18, wherein the compression mechanism is connected to the second anchor member at a fourth attachment point, and as the first anchor member applies the first mechanical force to the compression mechanism, the third attachment point and the fourth attachment point are configured to move apart relative to each other.
